SEQUENCE LISTING (10 sequences)

SEQ ID NO: 1 - protein, 5 aa, homo sapien
  VARIANT 2, 3: Xaa = Any Amino Acid
LXXLL

SEQ ID NO: 2 - protein, 9 aa, Homo Sapien
  VARIANT 2, 3, 4, 6, 7, 8: Xaa = Any Amino Acid
LXXXIXXXL

SEQ ID NO: 3 - protein, 9 aa, Homo Sapien
  VARIANT 2, 3, 4, 6, 7, 8: Xaa = Any Amino Acid
LXXXIXXXI

SEQ ID NO: 4 - protein, 25 aa, Homo Sapien
GHSFADPASNLGLEDIIRKALMGSF

SEQ ID NO: 5 - protein, 39 aa, Homo Sapien
GTGLMTYRSQAVQEHASTNMGLEAIIRKALMGKYDQWEE

SEQ ID NO: 6 - protein, 26 aa, Homo Sapien
CHCEDFSKVSQNPILTSLLQITFGNG

SEQ ID NO: 7 - protein, 25 aa, Homo Sapien
CPSSHSSLTERHKILHRLLQEGSPS

SEQ ID NO: 8 - protein, 25 aa, Homo Sapien
GHGEDFSKVSQNPILTSLLQITGNG

SEQ ID NO: 9 - protein, 22 aa, Homo Sapien
TNMGLEAIIFKALMGKYDQWEE

SEQ ID NO: 10 - protein, 11 aa, Homo Sapien
MKKGHHHHHHG

(19) United States
(12) Patent Application Publication
(10) Pub. No.: US 2005/0227220 A1
(43) Pub. Date: Oct. 13, 2005

(54) Compositions and Methods for Evaluating and Designing Nuclear Receptor Ligands that Modulate Co-Regulator Affinity

(76) Inventors: Steven Gerard Blanchard, Durham, NC (US); Thomas B. Stanley, Durham, NC (US); Julie Beth Stimmel, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/511,055

(22) PCT Filed: Apr. 11, 2003
(86) PCT No.: PCT/US03/11055

§ 371 Date: Oct. 12, 2004

Related U.S. Application Data
(60) Provisional application No. 60/372,524, filed on Apr. 12, 2002.

(51) Int. Cl. C12Q 1/00; G01N 33/53; G01N 33/567; C07K 14/47

(52) U.S. Cl. 435/4; 530/324; 530/326; 435/7.2

(57) ABSTRACT

Isolated corepressor peptides derived from a nuclear receptor interacting motif of corepressor Nuclear Receptor Corepressor (NCoR) or Silencing Mediator of Retinoid and Thyroid receptors (SMRT) are provided which binds to nuclear receptor ligand binding domains. Methods of using these peptides to evaluating selectivity of a ligand for a nuclear receptor subtype and quantitatively profiling selectivity of a test compound for a nuclear receptor are also provided.

### FIELD OF THE INVENTION

The present invention relates to the use of coactivator-derived peptides and/or fragments containing the characteristic LXXLL (SEQ D NO:1) motif and co-repressor-derived peptides and/or fragments containing the LXXXIXXXL (SEQ ID NO:2) motif as models to measure the effect of compounds on the binding/displacement of co-activators/co-repressors to nuclear receptors. It has now been found that different agonists increase the binding to a number of peptides and/or fragments derived from coactivators, but have varied effects on co-repressor binding to three PPAR subtypes, PPARα, PPARδ and PPARγ. The molecular mechanism for these effects is related to differences in how various ligands occupy the ligand binding pockets of these nuclear receptor PPAR subtypes, resulting in altered presentation of the co-activator/co-repressor binding surface. Accordingly, different compound classes are expected to have different receptor specificities and biological effects depending on the co-activator/co-repressor context of the target tissue. The compositions and methods of the present invention are useful in predicting in vivo effects of different classes of nuclear receptor ligands and designing new ligands that specifically modulate cofactor affinities for selected nuclear receptors.

### BACKGROUND OF THE INVENTION

Peroxisome Proliferator-Activated Receptors (PPARs) are transcription factors of the nuclear receptor family believed to be involved in the regulation of glucose, lipid and cholesterol levels. Willson, T. M., et al., The PPARs: from orphan receptors to drug discovery. Journal of Medicinal Chemistry, 2000. 43(4): p. 527-50. Natural ligands for PPARs are believed to be fatty acids and their derivatives. There are three subtypes of PPARs: PPARα (NR1C1) PPARδ (NR1C2) and PPARγ (NR1C3). All three subtypes share a domain organization common to other nuclear receptors with a highly conserved N-terminal DNA binding domain and a conserved C-terminal ligand binding domain (LBD). Due to their important role in multiple metabolic pathways, PPARs are targets for a number of drug therapies. PPARα is the target for lipid lowering fibrate drugs, while PPARγ is the target for antidiabetic drugs of the thiazolidinedione (TZD) class which include troglitazone, pioglitazone and rosiglitazone. Issemann, I. and S. Green, Activation of a member of the steroid hormone receptor superfamily by peroxisome proliferators. Nature, 1990. 347(6294): p. 645-50; Lehmann, J. M., et al., An antidiabetic thiazolidinedione is a high affinity ligandfor peroxisome proliferator-activated receptor gamma (PPAR gamma). Journal of Biological Chemistry, 1995. 270(22): p. 12953-6.

The PPAR subtypes bind to DNA as heterodimeric complexes with retinoid X receptors (RXR), that are receptors for 9-cis-retinoic acid (9cRA). The three RXR subtypes (α, β, γ) bind DNA and activate transcription as homodimers in response to 9cRA, but also serve as heterodimeric partners to more than 10 nuclear receptors that include liver X receptors (α, β), farnesoid X receptor, retinoid acid-related receptors (α, β, γ) and liver receptor homologue-1. Thus, the hormone signaling pathways controlled by these receptors can be modulated by two distinct ligands.

Ligand effects on gene activation are mediated through ligand binding of coactivator or co-repressor proteins to nuclear receptor. Robyr, D., A. P. Wolffe, and W. Wahli, Nuclear hormone receptor coregulators in action: diversity for shared tasks. Molecular Endocrinology, 2000. 14(3): p. 329-47. An increasing number of co-activator proteins have been identified for nuclear receptors. A number of these have been implicated in regulation of PPARs. For example SRC/p160 family and CBP/p300 have inherent histone acetylase activity that remodels chromatin structure. Zhu, Y., et al., Cloning and identification of mouse steroid receptor coactivator-1 (mSRC-1), as a coactivator of peroxisome proliferator-activated receptor gamma. Gene Expression, 1996. 6(3): p. 185-95; DiRenzo, J., et al., Peroxisomeproliferator-activated receptors and retinoic acid receptors differentially control the interactions of retinoid X receptor heterodimers with ligands, coactivators, and corepressors. Molecular & Cellular Biology, 1997. 17(4): p. 2166-76; Dowell, P., et al., p300functions as a coactivatorfor the peroxisome proliferator-activated receptor alpha. Journal of Biological Chemistry, 1997. 272(52): p. 33435-43. Co-activators such as TRAP220 interact with the basal transcriptional machinery. Zhu, Y., et al., Isolation and characterization of PBP, a protein that interacts with peroxisome proliferator-activated receptor. Journal of Biological Chemistry, 1997. 272(41): p. 25500-6; Yuan, C. X., et al., The TRAP220 component of a thyroid hormone receptor-associated protein (TRAP) coactivator complex interacts directly with nuclear receptors in a ligand-dependent fashion. Proceedings of the National Academy of Sciences of the United States of America, 1998. 95(14): p. 7939-44. Other co-activators that have been identified, whose mechanism of activation is unclear include: PGC-1 and RAP250/ASC-2. Puigserver, P., et al., A cold-inducible coactivator of nuclear receptors linked to adaptive thermogenesis. Cell, 1998. 92(6): p. 829-39; Caira, F., et al., Cloning and characterization of RAP250, a novel nuclear receptor coactivator. Journal of Biological Chemistry, 2000. 275(8): p. 5308-17; Lee, S. K., et al., A nuclearfactor, ASC-2, as a cancer-amplified transcriptional coactivator essentialfor ligand-dependent transactivation by nuclear receptors in vivo. Journal of Biological Chemistry, 1999. 274(48): p. 34283-93; Zhu, Y., et al., Isolation and characterization of peroxisome proliferator-activated receptor (PPAR) interacting protein (PRIP) as a coactivator for PPAR. Journal of Biological Chemistry, 2000. 275(18): p. 13510-6. The majority of these coactivators possess one or more nuclear receptor interaction domains composed of a conserved LXXLL motif that mediate the interaction of the co-activator with the ligand binding domain of the nuclear receptor. Darimont, B. D., et al., Structure and specificity of nuclear receptor-coactivator interactions. Genes & Development, 1998. 12(21): p. 3343-56; Nolte, R. T., et al., Ligand binding and co-activator assembly of the peroxisome proliferator-activated receptor-gamma. Nature, 1998. 395(6698): p. 137-43. In the unliganded state or, in some cases, the presence of antagonists, many nuclear receptors are complexed with corepressor proteins which repress transcription, apparently via an associated histone-deacetylase activity. The addition of agonist ligands promotes the dissociation of the corepressor and the binding of co-activator proteins resulting in an enhancement in the basal level of transcription of specific genes. Chen, J. D. and R. M. Evans, A transcriptional co-repressor that interacts with nuclear hormone receptors. Nature, 1995. 377(6548): p. 454-7. Altered interactions of nuclear receptors with co-repressor proteins have been implicated in a number of disease states and pathologies such as thyroid hormone resistance syndromes, promyelocytic leukemia and tamoxifen-resistant breast cancer. Yoh, S. M., V. K. K. Chatterjee, and M. L. Privalsky, Thyroid Hormone Resistance Syndrome Manifests as an Aberrant Interaction between Mutant T3 Receptors and Transcriptional Corepressors. Mol Endocrinol, 1997. 11(4): p. 470-480; Lavinsky, R. M., et al., Diverse signaling pathways modulate nuclear receptor recruitment of N-CoR and SMRT complexes. Proceedings of the National Academy of Sciences of the United States of America, 1998. 95(6): p. 2920-5; Grignani, F., et al., Fusion proteins of the retinoic acid receptor-alpha recruit histone deacetylase in promyelocytic leukaemia. Nature, 1998. 391(6669): p. 815-8; Yamamoto, Y., et al., The Tamoxifen-responsive Estrogen Receptor alpha Mutant D351Y Shows Reduced Tamoxifen-dependent Interaction with Corepressor Complexes. J. Biol. Chem., 2001. 276(46): p. 42684-42691.

Two of the better characterized co-repressors are Nuclear Receptor CoRepressor (NCOR) and Silencing Mediator for Retinoid and Thyroid receptors (SMRT). The nuclear receptor binding sites on these co-repressors have been localized to homologous domains within the C-ternini of both NCOR and SMRT. Hu, X. and M. A. Lazar, The CoRNR motif controls the recruitment of corepressors by nuclear hormone receptors. Nature, 1999. 402(6757): p. 93-6; Perissi, V., et al., Molecular determinants of nuclear receptor-corepressor interaction. Genes & Development, 1999. 13(24): p. 3198-208; Nagy, L., et al., Mechanism of corepressor binding and release from nuclear hormone receptors. Genes & Development, 1999. 13(24): p. 3209-16. Motifs analogous to the LXXLL (SEQ ID NO:1) found for coactivator binding have been found for co-repressors. Multiple (I/L)XX(I/V)I motifs have been identified in the C-terminal region of both NCOR and SMRT which mediate the interactions with nuclear receptors. Hu, X. and M. A. Lazar, The CoRNR motif controls the recruitment of corepressors by nuclear hormone receptors. Nature, 1999. 402(6757): p. 93-6; Nagy, L., et al., Mechanism of corepressor binding and release from nuclear hormone receptors. Genes & Development, 1999. 13(24): p. 3209-16; Webb, P., et al., The nuclear receptor corepressor (N-CoR) contains three isoleucine motifs (I/LXXII) that serve as receptor interaction domains (IDs). Molecular Endocrinology, 2000. 14(12): p. 1976-85; Cohen, R. N., et al., The Nuclear Corepressors Recognize Distinct Nuclear Receptor Complexes. Mol Endocrinol, 2000. 14(6): p. 900-914. The co-repressor motif has also been suggested to be extended and to include an additional hydrophobic residue to produce a LXXXIXXX(L/I) (SEQ ID NO:2/3) motif Perissi, V., et al., Molecular determinants of nuclear receptor-corepressor interaction. Genes & Development, 1999. 13(24): p. 3198-208. Binding and structural data indicate that the co-repressor motif is an extended LXXXIXXXL (SEQ ID NO:2) motif. Xu, H. E., et al Structural basis for antagonist-mediated recruitment of nuclear co-repressors by PPARα. Nature, 2002. 415(6873) p. 813-817. Alanine scanning mutagenesis of the SMRT co-repressor motif binding to both PPARα and TRβ indicate that L+1, I+5 and L+8 are critical to receptor binding. The structure of the PPARα LBD in the presence of a peptide from the co-repressor SMRT has been determined and indicates that the co-activator and co-repressor binding sites overlap. Xu, H. E., et al Structural basisfor antagonist-mediated recruitment of nuclear co-repressors by PPARα. Nature, 2002. 415(6873) p. 813-817. In this structure, the co-repressor motif forms a three-turn alpha helix that prevents the arrangement of the C-terminal AF2 helix into an active conformation. Therefore, correct positioning of the AF2 helix by agonist ligands and co-activator proteins is essential for nuclear receptor activation. Displacement of the AF2 helix by antagonists may facilitate the interaction with co-repressor proteins and is one potential mechanism for repression of nuclear receptor activity.

The structures of the ligand binding domains of all three PPAR receptor subtypes have been solved and indicate that key residues in the ligand binding pockets are responsible for ligand selectivity among subtypes and may be responsible for the specific pharmacologies of the different receptor subtypes. Gampe, R. T., Jr., et al., Asymmetry in the PPARgamma/RXRalpha crystal structure reveals the molecular basis of heterodimerization among nuclear receptors. Molecular Cell, 2000. 5(3): p. 545-55; Nolte, R. T., et al., Ligand binding and co-activator assembly of the peroxisome proliferator-activated receptor-gamma. Nature, 1998. 395(6698): p. 137-43; Xu, H. E., et al., Structural determinants of Ligand Binding Selectivity between the Peroxisome Proliferator-activated Receptors. PNAS, 2001. 98(24): p. 13919-13924; Xu, H. E., et al., Molecular recognition of fatty acids by peroxisome proliferator-activated receptors. Molecular Cell, 1999. 3(3): p. 397-403; Kliewer, S., et al., Differential Expression and Activation of a Family of Murine Peroxisome Proliferator-Activated Receptors. PNAS, 1994. 91(15): p. 7355-7359. Novel tyrosine-based PPARγ agonists that are potent insulin sensitizers have been disclosed. Henke, B. R., et al., N-(2-Benzoylphenyl)-L-tyrosine PPARgamma agonists. 1. Discovery of a novel series of potent antihyperglycemic and antihyperlipidemic agents. Journal of Medicinal Chemistry, 1998. 41(25): p. 5020-36; Collins, J. L., et al., N-(2-Benzoylphenyl)-L-tyrosine PPARgamma agonists. 2. Structure-activity relationship and optimization of the phenyl all ether moiety. Journal of Medicinal Chemistry, 1998. 41(25): p. 5037-54; Cobb, J. E., et al., N-(2-Benzoylphenyl)-L-tyrosine PPARgamma agonists. 3. Structure-activity relationship and optimization of the N-aryl substituent. Journal of Medicinal Chemistry, 1998. 41(25): p. 5055-69; Brown, K. K., et al., A novel N-aryl tyrosine activator of peroxisome proliferator-activated receptor-gamma reverses the diabetic phenotype of the Zucker diabetic fatty rat. Diabetes, 1999. 48(7): p. 1415-24. Many of these tyrosine-based compounds are highly selective for PPARγ in binding and transient transfection assays.

Due to the important roles of the nuclear receptor/corepressor interaction in a number of pathologies and disease states, an understanding of this interaction is essential for understanding nuclear receptor biology and the design of nuclear receptor targeted therapies.

### SUMMARY OF THE INVENTION

An object of the present invention is to provide co-repressor peptides and/or fragments thereof useful in combination with co-activator peptides and, where applicable, a heterodimeric partner, to evaluate the profile of a ligand with nuclear receptors.

Another object of the present invention is to provide a method for evaluating the profile of a ligand for an individual nuclear receptor, which comprises assessing a ligand's ability to increase or inhibit the binding of co-activator peptides and/or fragments thereof, and assessing the ability of the same ligand to increase or inhibit the binding of co-repressor peptides and/or fragments thereof, and, wherein applicable, to increase or inhibit the binding of a heterodimeric partner to the nuclear receptor.

Another object of the present invention is to provide a method for evaluating the selectivity of a ligand across multiple nuclear receptors which comprises assessing a ligand's ability to increase or inhibit the binding of co-activator peptides and assessing the ability of the same ligand to increase or inhibit the binding of co-repressor peptides and/or fragments thereof and, wherein applicable, to increase or inhibit the binding of a heterodimeric partner to the nuclear receptor. The profile of the ligand is determined on each of the nuclear receptors of interest, and comparison of the individual profiles thus obtained for each of the receptors provides an indication of the ligand's selectivity. Determination of the selectivity of a ligand for the three PPAR subtypes is an example of this method.

Yet another object of the present invention provides for structure-based design and/or identification of ligands selective for a particular nuclear receptor based upon the profiling techniques and co-repressor peptides and/or fragments thereof described herein.

### BRIEF DESCRIPTION OF THE DRAWINGS

FIG. 1 illustrates the results of a fluorescence polarization assay for ligand modulation of nuclear receptor/cofactor peptide affinities.

FIG. 2 shows the structures of several PPAR compounds.

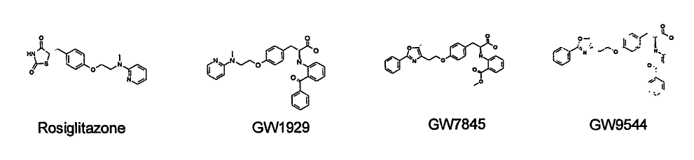

FIG. 3 illustrates the results of a ligand-dependent modulation of the binding of co-repressor peptides to PPAR LBD subtypes.

FIG. 4 illustrates the results of a GW1929 modulation of PPARγ cofactor complexes.

FIG. 5 illustrates the effect of ligands on coactivator and corepressor binding to PPAR subtypes determined by FRET.

FIG. 6 shows the characterization of ligand effects on PPAR/cofactor interactions in a cell-based assay.

FIG. 7 illustrates the results of a fluorescence energy transfer assay for LXR.

### DETAILED DESCRIPTION OF THE INVENTION

Nuclear receptor co-repressors appear to exert their repressive effects on gene activation by binding to unliganded nuclear receptors, with a concomitant increase in histone deacetylase activity, and by preventing the binding of co-activator proteins. With thyroid and retinoid receptors, natural ligands are believed to activate transcription by causing dissociation of the co-repressor, and association of co-activator, proteins. It has now been found that peptides derived from nuclear receptor interacting motifs of the corepressors Nuclear Receptor Corepressor (NCoR) and Silencing Mediator of Retinoid and Thyroid receptors (SMRT) are able to bind a number of nuclear receptor ligand binding domains including all three PPAR subtypes, PPARγ, PPARα, and PPARδ. In a preferred embodiment, these co-repressor peptides comprise
GHSFADPASNLGLEDIIRKALMGSF (NCoR 2251-2275, NCoR ID-C)(SEQ ID NO:4) and GTGLMTYRSQAVQEHASTNMGLEAIIRKALMGKYDQWEE (SMRT 2321-2361, SMRT ID-C)(SEQ ID NO:5) or a fragment thereof.

For purposes of the present invention, by the term “fragment” it is meant a polypeptide shorter in amino acid sequence than the co-repressor peptides taught herein but which exhibits similar biological activities to these co-repressor peptides.

Using these peptides and/or fragments derived from these co-repressors, it is now possible to characterize a ligand's profile with different nuclear receptors.

By “ligand profile” as used herein it is meant a group of characteristics of the ligand determined by various assays used to assess or evaluate its selectivity for a nuclear receptor. Examples of assays used to generate ligand profiles in accordance with the present invention include, but are not limited to, assessment of a ligand's ability to increase or inhibit the binding of co-activator peptides and/or fragments thereof, assessment of the ability of the same ligand to increase or inhibit the binding of co-repressor peptides and/or fragments thereof, assessment of the ability of a ligand to increase or inhibit the binding of a heterodimeric partner to the nuclear receptor, assessment of the activity of a ligand in standard nuclear receptor-ligand binding assays, and biological activity of the ligand in cell-based reporter assays and/or disease-specific cell-based assays. Standard nuclear receptor-ligand binding assays, as well as cell-based reporter assays and disease-specific cell-based assays can be performed routinely in accordance with well known procedures.

Examples of nuclear receptors, the ligand profiles for which can be characterized in accordance with the present invention, include, but are not limited to, the PPAR subtypes PPARα, PPARγ, and PPARδ, the estrogen-related receptors ERRα, ERRβ, and ERRγ, farnesoid X receptor (FXR), the liver X receptors LXRα, and LXRβ, the retinoid acid-related orphan receptors RORα, RORβ, and RORγ, liver receptor homologue (LRH-1) and CAR.

Ligands of nuclear receptors promote the association of co-activator peptides as expected. However, the same ligands have varied effects on the binding of co-repressor peptides. For example, as demonstrated herein, some members of a class of L-tyrosine-based compounds designed as selective agonists for PPARγ reduce the affinity for corepressor peptides on PPARγ, but increase the affinity for the same co-repressor peptides on PPARδ and in some cases PPARα. The structural basis for these observations has now been found to result from small differences in the ligand binding pockets of the three PPAR subtypes that are perturbed differentially by individual ligands and result in altered presentations of the overlapping co-activator/co-repressor binding surfaces. These differences in ligand binding pockets and the altered presentations of the overlapping co-activator/co-repressor binding surfaces can now be used to design selective ligands of nuclear receptors useful in treatment of a number of different diseases.

The biological effects of a ligand on a specific nuclear receptor depend not only on the intrinsic affinity of the ligand for the receptor but also the co-regulator context of the target cell. The co-regulator context of the target cells is related to both the types of co-activators and co-repressors as well as the relative levels/concentrations of co-activators and co-repressors in the target cells. Levels of co-activators and co-repressors are believed to vary significantly among cell and tissue types. Previous studies have used peptides and fragments of coactivators to profile the effects of ligands on co-activator binding. Bramlett, K. S., Y. Wu, and T. P. Burris, Ligands specify coactivator nuclear receptor (NR) box affinity for estrogen receptor subtypes. Molecular Endocrinology, 2001. 15(6): p. 909-22; Wong, C. W., B. Komm, and B. J. Cheskis, Structure-function evaluation of ER alpha and beta interplay with SRC family coactivators. ER selective ligands. Biochemistry, 2001. 40(23): p. 6756-65; Warnmark, A., et al., Differential Recruitment of the Mammalian Mediator Subunit TRAP220 by Estrogen Receptors ERalpha and ERbeta J. Biol. Chem., 2001. 276(26): p. 23397-23404. In the present invention, co-repressor peptides are provided thus enabling a more complete profile of a compound to be obtained by assessing not only its ability to bind co-activators, but also to displace or bind co-repressors.

In the present invention, biophysical techniques including fluorescence polarization (FP), fluorescent resonance energy transfer (FRET) and surface plasmon resonance (SPR) are used to measure the binding of purified ligand binding domains of a number of nuclear receptors to peptides derived from relevant co-activators and co-repressors. Using these techniques, binding affinities of the nuclear receptor/coregulator motif interactions can be rapidly determined and the quantitative effects of specific ligands on these interactions can be deduced. Results from these biophysical techniques are consistent with previous cell-based and co-precipitation assays for nuclear receptor corepressor interactions. Accordingly, these assays serve as models for biological interactions of nuclear receptors and their ligands and permit quantitative assessment of ligand induced receptor preferences for different coregulators and the apparent ligand selectivity in different coregulator environments.

Fluorescence polarization was used to determine the binding affinities of various nuclear receptor ligand binding domains for co-repressor peptides derived from the C-terminal nuclear receptor binding site(s) of NCoR and SMRT. These co-repressor -derived peptides were N-terninally labeled with fluorescein and contain the LXXXIXXXL (SEQ ID NO:2) motif essential for nuclear receptor binding. Increasing concentrations of all eight ligand binding domains tested increased the fluorescence polarization of the NCoR ID-C and SMRT ID-C peptides. The affinities of these peptides for ligand binding domains of eight nuclear receptors are summarized in Table I.

TABLE I

| | NcoR | | SMRT | |
|---|---|---|---|---|
| Receptor LBD | K_{D} (μM) | Error | K_{D} (μM) | Error |
| TR-β | 1.3 | 0.03 | 0.74 | 0.04 |
| RAR-α | 2.0 | 0.1 | 2.1 | 0.2 |
| PPAR-α | 6.1 | 0.12 | 6.1 | 0.2 |
| PPAR-γ | 1.3 | 0.03 | 1.8 | 0.1 |
| PPAR-δ | 5.7 | 0.2 | 7.7 | 0.6 |
| RXR-α | 25.0 | 0.14 | 20.0 | 0.2 |
| RXR-α ΔAF2 | 0.39 | 0.03 | 0.25 | 0.02 |
| ER-β | 37.0 | 5.0 | 30.0 | 4.0 |

As shown in this Table, the nuclear receptor ligand binding domains from TRβ, PPARγ and RARα exhibited the highest affinities (0.7 to 2 μM for both the NCoR and SMRT peptides. The ligand binding domains from PPARα and PPARδ exhibited affinities in the 5-6 μM range. In contrast, the ligand binding domain from RXRα had the weakest affinity (˜20 μM) for both co-repressor peptides. A modified RXRα ligand binding domain wherein the AF2 region is deleted (RXRαΔAF2), however, has approximately 100-fold greater affinity for the co-repressor peptides. This quantitative evidence is consistent with previous observations that only an AF2-truncated RXRα showed measurable corepressor binding and that in general the AF2 domain appears to sterically compete with co-repressor binding. Zhang, X., et al., A Nuclear Receptor Corepressor Modulates Transcriptional Activity of Antagonist-Occupied Steroid Hormone Receptor. Mol Endocrinol, 1998. 12(4): p. 513-524; Chen, J. D. and R. M. Evans, A transcriptional co-repressor that interacts with nuclear hormone receptors. Nature, 1995. 377(6548): p. 454-7; Horlein, A. J., et al., Ligand-independent repression by the thyroid hormone receptor mediated by a nuclear receptor co-repressor. Nature, 1995. 377(6548): p. 397-404. The weak affinity of the co-repressor peptides for ERβ is consistent with previous reports that unliganded steroid receptors have very weak affinity for corepressors. Lavinsky, R. M., et al., Diverse signaling pathways modulate nuclear receptor recruitment of N-CoR and SMRT complexes. Proceedings of the National Academy of Sciences of the United States of America, 1998. 95(6): p. 2920-5; Zhang, X., et al., A Nuclear Receptor Corepressor Modulates Transcriptional Activity of Antagonist-Occupied Steroid Hormone Receptor. Mol Endocrinol, 1998. 12(4): p. 513-524. Binding affinities to NCOR ID-C and SMRT ID-C peptides for individual ligand binding domains were virtually indistinguishable. Accordingly, receptor preferences for one co-repressor or another do not appear to be due to amino acid differences in the core C-terminal motif. As demonstrated herein, the co-repressor peptides clearly bind nuclear receptor ligand binding domains, and in particular PPAR ligand binding domains, with reasonable affinities such that they can be used to study the nuclear receptor/co-repressor interaction.

The quantitative profiling techniques of the present invention were used to assess the effects of ligand classes on coregulator binding to the three PPAR subtypes, since all three of these receptors are of intense interest for their biological role in several disease states and as targets of drug therapies.

A linked equilibrium between co-activator/co-repressor binding and the binding of ligand is well established, such that the binding of ligand can alter the binding affinity for co-activator/co-repressor and visa versa. Gee, A. C., et al., Coactivator Peptides Have a Differential Stabilizing Effect on the Binding of Estrogens and Antiestrogens with the Estrogen Receptor. Mol Endocrinol, 1999. 13(11): p. 1912-1923. Therefore, tests were performed to determine whether the co-repressor peptides could be used to replicate the ligand-induced effects observed for the nuclear receptor/co-repressor interaction in cellular and in vivo assays. Such tests were performed quantitatively by measuring the effect of an excess of ligand on the binding affinity of the co-repressor peptide. Peptides from TRAP220 and SRC-1 containing the LXXLL (SEQ ID NO:1) motif critical for nuclear receptor binding were used as co-activator peptides. It is preferred in this assay that the co-activator peptides comprise CHCEDFSKVSQNPILTSLLQITFGNG (TRAP220 575-599) (SEQ ID NO:6) and CPSSHSSLTERHKILHRLLQEGSPS (SRC-1 (LCD2) 676-700) (SEQ ID NO:7). Increasing concentrations of the TRβ LBD increased the polarization of the fluorescent SRC and NCOR peptides, indicative of binding of the peptide to the receptor. Addition of the agonist ligand T3 increased the affinity of the receptor-SRC-1 interaction 50-fold to 130 nM, but weakened the affinity of the receptor-NCOR ID-C interaction 9-fold to 6.2 μM. In the absence of ligand, the TRβ ligand binding domain had a 10-fold greater affinity for the corepressor peptide than coactivator peptide. Yet in the presence of the agonist ligand T3, this preference was reversed with the TRβ ligand binding domain having a 48-fold greater affinity for coactivator peptide than co-repressor peptide. Similar behavior was observed with PPARγ; the TZD, rosiglitazone, increased the affinity of receptor for TRAP220 approximately 3-fold and reduced the affinity of receptor for NCOR approximately 4-fold. The L-tyrosine-based compound GW1929 also increased the affinity of receptor for the TRAP220 co-activator peptide 5-fold and reduces the affinity of receptor for NCOR co-repressor peptide 5-fold. These results quantitatively demonstrate that ligand-induced changes in affinity for coregulator peptides result in exchange of co-repressor for co-activators.

The effects of rosiglitazone and GW1929 (described in detail in WO 97/31907) on binding of peptides to the PPARδ ligand binding domains were determined. Both of these compounds are considered PPARγ specific compounds since they have much weaker affinities for PPARδ in cell-based functional and direct binding assays Willson, T. M., et al., The PPARs: from orphan receptors to drug discovery. Journal of Medicinal Chemistry, 2000. 43(4): p. 527-50. Rosiglitazone had little or no effect on PPARδ interaction with either the coactivator or corepressor peptide. Surprisingly, the PPAR agonist L-tyrosine compound GW1929 increased the affinity of PPARδ for the corepressor NCOR 12-fold but had little or no effect on the affinity of the co-activator TRAP220 to the same receptor. Thus, a compound that weakened the affinity of the corepressor peptide for PPARγ, had the opposite effect on PPARδ and increased the affinity of the PPARδ NCoR peptide interaction.

A surface plasmon resonance assay was used to further characterize the effects of PPAR ligands binding of peptides derived from the co-repressors NCOR and SMRT to the PPAR subtypes. Each peptide was biotinylated and immobilized on separate flow cells of a BlAcore sensor chip. Using a BIAcore instrument, fixed concentrations of each PPAR subtype ligand binding domain were incubated with varied concentrations of test compounds and injected over the peptide-derivatized sensor chip to determine the equilibrium binding response of the receptors for the immobilized peptides. Rosiglitazone and the tyrosine-based ligands GW1929 and GW7845 (each of which are described in detail in WO 97/31907) decreased the binding of the PPARγ ligand binding domain to the NCOR- and SMRT-derived co-repressor peptides. Although these compounds had significantly weaker binding affinities for PPARα and PPARδ, they were also able to affect the binding of the co-repressor peptides to these other PPAR subtypes. The tyrosine-based compounds increased binding of PPARδ ligand binding domain to both the NCOR and SMRT co-repressor peptides. With PPARα ligand binding domain, only GW7845 appeared to increase the binding to the NCOR and SMRT co-repressor peptides.

The quantitative effects of compounds on co-activator/co-repressor binding were profiled via a Fluorescence Resonance Energy Transfer (FRET) assay. Biotinylated coactivator fragments derived from CBP (amino acids 57-454) and a co-repressor peptide (NCoR ID-C) were labeled with europium-conjugated steptavidin. Biotinylated PPAR ligand binding domains were labeled with allophycocyanin-conjugated steptavidin. The effect of compounds on the binding of each PPAR subtype ligand binding domain to a fragment of CBP or the NCOR ID-C peptide were determined by varying the ligand concentration in the presence of 10 nM PPAR LBD and 4 different concentrations of CBP and NCoR. The data were simultaneously fit to an interaction model described in Example 6. The experimentally determined α value is the ratio of cofactor affinities in the presence and absence of a saturating concentration of ligand. This analysis permits a direct comparison of the effects of compounds, independent of cofactor concentration and differences in coregulator affinity for the individual receptors. The TZDs, rosiglitazone and pioglitazone, as well as tyrosine-based PPARγ agonists (GW1929, GW7845, GW2570 and GW9544, each of which are described in detail in WO 97/31907) and the PPARδ ligand, GW1516 increased the apparent affinity of all three PPAR subtypes for the co-activator fragment from CBP. Oliver, W. R., Jr., et al., A selective peroxisome proliferator-activated receptor delta agonist promotes reverse cholesterol transport. Proceedings of the National Academy of Sciences of the United States of America, 2001. 98(9): p. 5306-11. Thus, these various PPAR agonists each appear to exert consistent increases in affinity for the co-activator regardless of receptor subtype. In contrast, the ligands exerted varied PPAR subtype-dependent effects on the affinities for the co-repressor peptide. For example, TZD compounds and the PPARδ ligand, GW1516, decreased the affinity of all three subtypes for the co-repressor peptide. The tyrosine-based ligands decreased the affinity for PPARγ for the co-repressor peptide, but had little effect on, or modestly enhanced, the affinity of PPARδ for the co-repressor peptide. GW1929 showed the greatest effect. For PPARα, only GW7845 enhanced the binding of the co-repressor peptide. Thus, taken together, the experiments show overall profiles where, different compounds have varied effects on both the direction and magnitude of co-repressor binding. The effects on corepressor peptide binding observed by FRET are consistent with those observed in both the fluorescence polarization and surface plasmon resonance assays.

The effects of the coregulator environment on the apparent affinities and selectivities of various ligands were than examined. Experimentally determined pKs and the α values derived from the FRET assay were used to calculate the apparent affinity of the ligand for the receptor/cofactor complex (i.e. K/α). Although all three compounds differed in their potency, all were at least 100-fold selective for PPARγ in the absence of coregulator. In the presence of CBP, the affinity was increased for all three compounds on all PPAR subtypes, but the overall selectivity for PPARγ was maintained. Yet in the presence of NCoR, the selectivity profiles for the three compounds diverged significantly. The apparent affinity of rosiglitazone for the PPARγ/NCoR complex was approximately 700 nM, and more than 100 μM for the complex with PPARα and PPARδ. For GW1929, the apparent affinity for the PPARγ/NCoR complex was 150 nM, approximately 500 nM for PPARδ, but greater than 25 μM for PPARα. For GW7845, the affinity in the presence of NCoR was basically equipotent for PPARα, PPARγ and PPARδ, with apparent affinities of 340 nM, 130 nM and 290 nM, respectively. Thus, in the presence of NCoR, rosiglitazone is a highly selective compound for PPARγ, GW1929 is selective for PPARγ and PPARδ, and GW7845 demonstrates almost no subtype selectivity. Therefore, the activity and apparent selectivity of compounds and hence the biological effects of compounds are highly dependent on the co-activator/co-repressor context.

The activation of the PPAR subtypes also occurs though binding to response elements as a heterodimeric complex with RXR. The quantitative effects of compounds on RXR binding to PPARs were profiled via a Fluorescence Resonance Energy Transfer (FRET) assay. Biotinylated RXRα LBD was labeled with europium-conjugated steptavidin and biotinylated PPAR subtype ligand binding domains were labeled with allophycocyanin-conjugated steptavidin. The effects of compounds on the binding of each PPAR subtype ligand binding domain to RXRα LBD were determined by varying the ligand concentration in the presence of 10 nM PPAR LBD and 4 different concentrations of RXR. The data were simultaneously fit to the interaction model described in Example 6. All ligands evaluated promoted the association between the three PPAR subtypes and RXR to varying extents. Rosiglitazone was most effective at promoting the association of RXR to PPARγ. Pioglitazone was equally effective at promoting the association of RXR with all three PPAR subtypes. Furthermore, GW1516, GW1929, GW7845, farglitazar and GW9544 were most effective at promoting the association of PPARδ with RXR. Thus, from these experiments, it is clear that ligand binding to a nuclear receptor can affect the apparent selectivity of a heterodimeric partner for the nuclear receptor. This interaction is an important component of the overall profile of ligands for nuclear receptors that form heterodimeric complexes, such as PPARs and RXR.

Mammalian two-hybrid assays were used to examine the ability of the tyrosine-based class of PPAR ligands to promote association or dissociation of the co-activator CBP and the corepressor NCoR in the context of a cellular environment. The assays for the three PPAR subtypes used full length human PPARα, PPARγ2 or PPARδ fused to the activation domain of VP16 and previously identified interaction domains of CBP and NCoR fused to the DNA binding domain of GAL4. Each assay was optimized with the intent to demonstrate ligand-dependent association or dissociation to full length PPARs by coregulators. Thus, the total fold activations were not as high as described in other assays. All ligands evaluated effectively promoted the association of CBP to full length PPAR subtypes. All PPAR subtypes also interacted with corepressor interaction domain (2239-2300) containing NCoR ID-C and modulation of co-repressor interaction was seen with GW6471, pioglitazone and GW1516 with PPARα, PPARγ and PPARδ, respectively. Neither of the PPAR subtypes interacted with a NCoR fragment (2012-2103) that contained NCoR ID-N. This is consistent with the results obtained using other biophysical techniques. A larger fragment of NCoR (1944-end) that contained both NCoR co-repressor interaction domains, NCoR ID-C and NCoR ID-N, demonstrated similar characteristics to those found for NCoR (2239-2300). Accordingly, for PPARγ, the tyrosine-based ligands GW1929 and GW7845 promoted association with co-activator CBP approximately 3-fold and promoted dissociation with co-repressor NCoR approximately 3-fold. Evaluation with PPARδ demonstrated that both ligands promoted association with CBP approximately 2-fold, though only GW1929 as able to slightly promote association with NCoR. Similar results with CBP were obtained with these ligands and PPARα, though neither GW1929 nor GW7845 were able to promote association with NCoR. These results were consistent with the quantitative results obtained using the FRET assay. GW7845 (log α=0.14) was not as efficient as GW1929 (log α=0.52) in promoting NCoR association with PPARδ. Furthermore, the alpha value for the association of PPARα and NCoR with GW7845 was 0.16, similar to the value obtained with PPARδ where no significant association with NCoR was apparent in this assay. These results may be a reflection of the sensitivity of the cell-based versus cell-free assays. Overall, it is believed that the peptides derived from defined co-activator and co-repressor interaction domains are tools that can be used to model ligand-dependent coregulator modulation of PPAR interaction in a cellular context.

To understand the molecular basis for the unusual effect of tyrosine-based ligands on the affinity of co-repressor peptides, the cocrystal structure of PPARα bound to a SMRT corepressor peptide and the ligand GW7845 was determined. In the biophysical profiling assays, this ligand was found to decrease the affinity for co-repressor with PPARγ, but slightly increase the affinity for corepressor with PPARα. The overall structure of the ternary complex was found to be similar to that reported for the PPARα/SMRT structure in the presence of a tyrosine-based antagonist ligand, GW6471. Xu, H. E., et al., Structural basis for antagonist-mediated recruitment of nuclear co-repressors by PPARα. Nature, 2002. 415(6873) p. 813-817. The SMRT peptide forms a three-turn α-helix that traces on a groove formed by helices 3, 3′, 4 and 5, which also form the major part of the co-activator binding site. The PPARα/SMRT/GW7845 structure was overlaid with the previously described PPARα agonist structure with a tyrosine-based ligand in which the benzophenone group is replaced with a small vinylogous amide. Xu, H. E., et al., Structural determinants of Ligand Binding Selectivity between the Peroxisome Proliferator-activated Receptors. PNAS, 2001. 98(24): p. 13919-13924. Both ligands occupy the pocket in a similar mode, with their acidic headgroups pointing towards the active position of the AF2 helix. The hydrogen bond network between the ligand headgroup and residue Y464 (Y473 in PPARγ) in the AF2 helix has been shown to serve as a key “molecular switch” to activate PPARs. Nolte, R. T., et al., Ligand binding and co-activator assembly of the peroxisome proliferator-activated receptor-gamma. Nature, 1998.395(6698): p.137-43. The binding mode of GW7845 in PPARα appears to still allow its acidic group to hydrogen bond with the AF-2 helix in the absence of co-repressor peptide. However, the major differences between the PPARα agonist structure and the GW7845 corepressor structure reside in the so-called “benzophenone pocket” formed by the loop preceding the AF2 helix as well as helices 3, 6 and 10. Gampe, R. T., Jr., et al., Asymmetry in the PPARgamma/RXRalpha crystal structure reveals the molecular basis of heterodimerization among nuclear receptors. Molecular Cell, 2000. 5(3): p. 545-55. This benzophenone pocket is large in PPARγ, but is somewhat narrower in PPARα and substantially smaller in PPARδ. Thus, for the bulky benzophenone group to fit in, PPARα requires some rearrangement of the ligand binding pocket. In particular, steric collisions between the benzophenone group of GW7845 and Phe 273, Ala 254 and Leu 456 require reorientation of these residues to accommodate the benzophenone group. This rearrangement causes the ligand to push against the loop between helix 10 and AF2 that results in destabilization of the AF2 helix from its active position. The displacement of the AF2 helix from its active conformation opens up the co-activator-binding site for binding of the longer co-repressor helix.

These structural studies are indicative of the differences in the size of the benzophenone pocket in the three PPAR subtypes being the molecular basis for the differential effects of ligands on binding of co-repressors. Previous studies have shown that PPARs have substantially larger ligand binding pockets than other nuclear receptors, enabling PPARs to bind a wide variety of ligands. Activated structures of PPARγ and with rosiglitazone, the tyrosine-based ligand farglitazar (GW2570) as well as the dual PPARα/PPARγ tyrosine-based GW9544 have been described previously. Nolte, R. T., et al., Ligand binding and co-activator assembly of the peroxisome proliferator-activated receptor-gamma. Nature, 1998. 395(6698): p. 137-43; Gampe, R. T., Jr., et al., Asymmetry in the PPARgamma/RXRalpha crystal structure reveals the molecular basis of heterodimerization among nuclear receptors. Molecular Cell, 2000. 5(3): p. 545-55; Xu, H. E., et al., Structural determinants of Ligand Binding Selectivity between the Peroxisome Proliferator-activated Receptors. PNAS, 2001. 98(24): p. 13919-13924. These structures reveal that the “molecular switch” for activating the PPAR receptors is a hydrogen bond between Tyr 473 of the AF2 helix and the carboxyl headgroups of both rosiglitazone and the tyrosine-based compounds that orient the AF2 helix in an active conformation. The tyrosine GW7845 and GW1929 all have substituents not found in the TZDs, that insert into the “benzophenone pocket.” Gampe, R. T., Jr., et al., Asymmetry in the PPARgamma/RXRalpha crystal structure reveals the molecular basis of heterodimerization among nuclear receptors. Molecular Cell, 2000. 5(3): p. 545-55; Xu, H. E., et al., Structural determinants of Ligand Binding Selectivity between the Peroxisome Proliferator-activated Receptors. PNAS, 2001. 98(24): p. 13919-13924. Structural studies indicate that tyrosine-based ligands such as GW7845 can occupy the ligand pocket of PPARα and PPARδ, but only with significant alteration of the ligand binding pocket that likely results in their reduced affinity for PPARα or PPARδ. These ligands push against the helix 10 linker region and cause helix 12 to be unstructured such that it is not evident in the co-crystal of PPARα. Since the co-repressor motif and AF2 helix occupy the same space, they compete with one another for occupying the same position within the receptor. Therefore, as a consequence of the benzophenone group pushing against the AF2 helix linker and making the AF2 helix more mobile, the equilibrium is shifted towards co-repressor binding, resulting in either a modest increase or no effect on co-repressor affinity. This mechanism suggests that structure based-design that can be used to generate ligands with novel cofactor binding profiles. In contrast to other nuclear receptor ligands, tyrosine-based compounds containing substituents that occupy the benzophenone pocket also enhance the affinity for LXXLL(SEQ ID NO:1) coactivator motifs. The structure of PPARα complexed with GW7845 described herein shows that GW7845 would not prevent formation of the activating hydrogen bond between the ligand carboxyl group and Tyr 473 in the AF2 helix that leads to formation of a charge clamp and promotion of co-activator peptide binding. This finding contrasts with the previously described PPARα antagonist GW6471 which contains a bulky head-group that prevents the ligand from forming an activating hydrogen bond with the AF2 helix, resulting in reduced affinity for co-activator and enhanced affinity for co-repressors. Xu, H. E., et al Structural basis for antagonist-mediated recruitment of nuclear co-repressors by PPARα. Nature, 2002. 415(6873) p. 813-817. Nuclear receptor antagonists such as GW6471 in the case of PPARα, or tamoxifen and raloxifene in the case of estrogen receptors, have bulky substituents that protrude from the binding pocket. Brzozowski, A. M., et al., Molecular basis of agonism and antagonism in the oestrogen receptor. Nature, 1997. 389(6652): p. 753-8; Shiau, A. K., et al., The structural basis of estrogen receptor/coactivator recognition and the antagonism of this interaction by tamoxifen. Cell, 1998. 95(7): p. 927-37; Pike, A. C., et al., Structure of the ligand-binding domain of oestrogen receptor beta in the presence of a partial agonist and a full antagonist. EMBO Journal, 1999. 18(17): p. 4608-18. As a result, such ligands sterically prevent the AF2 helix from attaining an active conformation. (For a review see Bourguet, W., P. Germain, and H. Gronemeyer, Nuclear receptor ligand-binding domains: three-dimensional structures, molecular interactions and pharmacological implications. Trends in Pharmacological Sciences, 2000. 21(10): p. 381-8.) The structure of GW7845/PPARα shows that the ligand would not sterically prevent the AF2 helix from adopting an active conformation. Therefore, GW7845 could also enhance the binding of coactivators as is observed in binding studies described herein. In PPARγ, the benzophenone pocket is large enough to accommodate ligands capable of perturbing the helix 10 linker. These compounds promote dissociation of co-repressor and association of coactivators. Therefore, in order for tyrosine-based ligands such as GW7845 and GW1929 to bind to PPARα or PPARδ a rearrangement of the benzophenone pocket is required to affect the co-regulator binding surface and enhance the mobility of the AF2 helix. Therefore these ligands can enhance the affinity for both coactivators and corepressors.

While the primary biological effects of TZDs and the L-tyrosine-based compounds appear to be through the PPARγ receptor, the different co-activator/co-repressor profiles identified for these ligands are expected to result in significantly different pharmacological activities, depending on the co-regulator context of the target cell. This has been demonstrated for Selective Estrogen Receptor Modulators (SERMs). One SERM, tamoxifen, has been shown to be an antagonist in breast tissue and an agonist in uterine tissue. Recently, it has been demonstrated that tamoxifen induces a unique estrogen receptor conformation that allows for the binding of a tamoxifen-specific phage peptide. The ability of a ligand to increase binding of this peptide was -correlated with agonist activity in the uterus. Norris, J D, Paige, L A, Christensen, D J, Chang, C, Huacani, M R, Fan, D, Hamilton, P T, Fowlkes, D M and McDonnell, D P (1999) Peptide Antagonists of the Human Estrogen Receptor Science 285, 744-746. It can be concluded that this tamoxifen-specific peptide may mimic a co-regulator that is present in uterine tissue, but not in breast, tissue. This example reiterates that importance of considering the co-regulator context of particular tissues in assessing the biological effects of specific compounds and further demonstrates the importance of co-regulator profiling assays to characterize compounds and reveal correlations to biological activity. Future generations of nuclear receptor ligands will identified via the identification and characterization of compounds that modulate or promote the binding of desired co-regulators (a desired profile) that correlate with the desired pharmacological effect.

Using the biophysical techniques and co-repressor peptides of the present invention, the interaction of co-regulators (co-activator, co-repressor and heterodimeric receptor partner) with nuclear receptors can now be characterized, thus allowing a quantitative profiling of the effects of ligands on these interactions. As shown herein, the assays of the present invention can be used to identify ligands that have unexpected and previously unseen profiles in which a ligand can promote the binding of co-regulators and proteins.

The following nonlimiting examples are provided to further illustrate the present invention.

### EXAMPLES

### Example 1

Reagents

The following peptides were ordered from SynPep (Dublin, Calif.).

TRAP220 (amino acids 575-599; Genbank accession AAF98352) biotin-GHGEDFSKVSQNPILTSLLQITGNG (SEQ ID NO:8) SRC-1 (amino acids 676-700; Genbank accession U59302): biotin-CPSSHSSLTERHKILHRLLQEGSPS (SEQ ID NO:7) NCOR ID-C (amino acids 2251-2275; Genbank accession NP_{—}006302) biotin-GHSFADPASNLGLEDIIRKALMGSF (SEQ ID NO:4) Fluoroscein-GHSFADPASNLGLEDIIRKALMGSF (SEQ ID NO:4) SMRT ID-C (amino acids 2321-2361; Genbank accession XP_{—}045602) biotin-GTGLMTYRSQAVQEHASTNMGLEAIIRKALMGKYDQWEE (SEQ ID NO: 5) Fluoroscein-TNMGLEAIIFKALMGKYDQWEE (SEQ ID NO:9)

### Example 2

Expression and Purification of Nuclear Receptor Ligand Binding Domains

CREB binding protein (CBP, residues 57-454; Genbank accession S39162) and the human PPARα ligand binding domain (amino acids 192-468; Genbank accession A07689) the human PPARδ ligand binding domain (amino acids 139-441; Genbank accession L07592) and the human PPARγ ligand binding domain (amino acids 206-477; Genbank accession L40904) each containing the peptide sequence MKKGHHHHHHG (SEQ ID NO:10) were expressed in bacterial (BL21-DE3) cells using the plasmid vector pRSETA. Cells with expression plasmid were grown overnight at 20° C. in the presence of 50 μg/ml Carbenicillin. Cells were harvested and resuspended in 50 mM TRIS, pH 8.0, containing 250 mM NaCl, and lysed with a french press. The cells were centrifuged at 40,000 g for 30 minutes and the lysates were loaded on a Nickel-chelating sepharose fast flow (Pharmacia) column equilibrated with 50 mM TRIS, pH 8.0, containing 250 mm NaCl and 25 mM imidazole. Protein was eluted using a linear gradient from 25 mM to 500 mM imidazole and dialyzed against 50 mM TRIS, pH 7.0, containing 25 mM NaCl, 2 mM dithiothreitol (DTT) and 0.5 mM ethylene diamine tetraacetic acid (EDTA) The protein is then loaded on a SP-Sepharose fast flow (Pharmacia) column and eluted with a linear gradient from 25 mM to 500 mM NaCl. Proteins were aliquoted and stored at −80° C. until use.

The following nuclear receptor ligand binding domains were also purified using His-tag: RARα (amino acids 146-432; Genbank accession x06538) RXRα (amino acids 225-462 468; Genbank accession x52773) RXRαΔAF2 (amino acids 225-446; Genbank accession x52773), ERβ (amino acids 257-530; Genbank accession NM-001437), LXRα (amino acids 183-447; Genbank accession U22662) and LXRβ (amino acids 185-461; Genbank accession U07132).

### Example 3

Binding of Nuclear Receptor Ligand Binding Domains to Peptides by Fluorescence Polarization

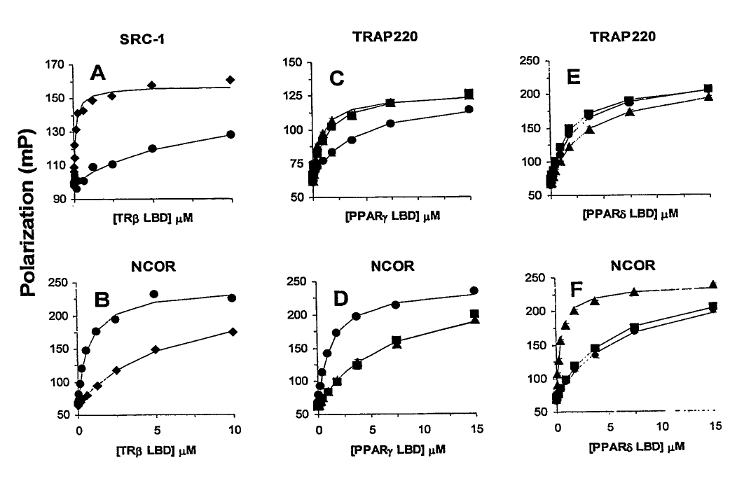

All experiments were conducted with buffer containing 10 mM HEPES, pH 7.4, containing 0.15 M NaCl, 3 mM EDTA, 0.005% polysorbate-20 and 5 mM DTT. Varied concentrations of receptor were incubated at room temperature with 10 nM of fluorescein-labeled NCOR ID-C or SMRT ID-C peptides. The fluorescence polarization values for each concentration of receptor were determined using a BMG PolarStar Galaxy fluorescence plate reader with 485 nm excitation and 520 nm emission filters. Binding isotherms were constructed and apparent KD values were determined by non-linear least squares fit of the data to an equation for a simple 1:1 interaction. FIG. 1 illustrates the results of a fluorescence polarization assay for ligand modulation of nuclear receptor/cofactor peptide affinities. Complex formation was monitored by fluorescence polarization as described in methods. Panel A shows the binding of Thyroid Receptor LBD to a peptide from SRC-1 is depicted with apparent affinities of 7.1±4.0 μM in the absence of ligand (●) and 0.13±0.02 μM in the presence of T3 (♦). Panel B shows the binding of Thyroid Receptor LBD to a peptide from NCoR is shown with apparent affinities of 0.72±0.08 μM in the absence of ligand (●) and 6.2±0.9 μM with T3 (♦). Panel C shows the binding of the PPARγ LBD to TRAP220 peptide is presented with apparent affinities of 3.9±0.5 μM without ligand (●), 0.79±0.09 μM with GW1929 (▴) and 1.5±0.2 μM with rosiglitazone (▪). Panel D shows the binding of PPARγ LBD to a NCOR peptide is presented with apparent affinities of 1.2±0.05 μM in the absence of ligand (●), 6.7±0.3 μM with GW1929 (▴) and 4.7±0.4 μM with rosiglitazone (▪). In Panel E the binding of PPARδ LBD to TRAP220 peptide is shown with apparent affinities of 2.3±0.1 μM without ligand (●), 3.4±0.2 μM with GW1929 (▴) and 1.8±0.1 μM with rosiglitazone (▪). Panel F shows the binding of PPARδ LBD to NCOR peptide with apparent affinities of 5.7±0.5 μM in the absence of ligand (●), 0.44±0.02 μM with GW1929 (▴) and 5.0±0.2 μM with rosiglitazone (▪). All assays were performed in duplicate and affinities are the average of at least two independent experiments.

### Example 4

Effect of Compounds on Coactivator/Corepressor Peptide Binding Measured by Surface Plasmon Resonance

The binding of co-activator and co-repressor peptides with PPAR ligand binding domains and the effects of compounds on these interactions were determined by surface plasmon resonance using the Biacore 3000. Approximately 300 resonance units (RU) of biotinylated peptides were immobilized to individual flow cells of a streptavidin chip. All experiments were conducted at 25° C. at a flow rate of 10 μl/minute with a buffer containing 10 mM HEPES, pH 7.4, containing 0.15 M NaCl, 3 mM EDTA and 0.005% polysorbate-20. The effect of compounds was determined by pre-incubating test compounds (10 μM) with 5 μM PPARα ligand binding domain or 2.5 μM PPARγ or PPARδ ligand binding domains for at least 30 minutes and then injecting 20 μl at 10 μl/minute. Binding was determined by measuring the equilibrium binding response 10 seconds before the end of the association phase minus the response from a flow cell with no immobilized peptide.

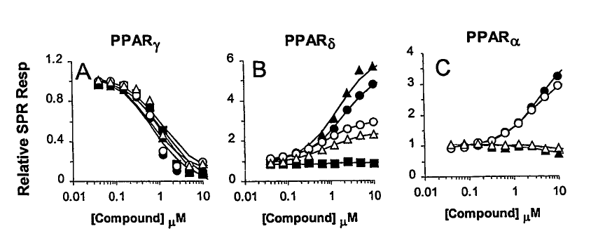

FIG. 3 illustrates the results of a ligand-dependent modulation of the binding of co-repressor peptides to PPAR LBD subtypes. The effects of GW1929, rosiglitazone and GW7845 on PPAR LBD subtypes binding to co-repressor peptides were characterized using a surface plasmon resonance (SPR) as described in methods. The relative equilibrium SPR response (response in presence of compound/response in absence of compound) of GW1929 (▴), GW7845 (●) or rosiglitazone (▪) on 2 μM receptor binding to NCOR ID-C (closed symbols), or SMRT ID-C (open symbols) is shown. Each point is the average of three injections.

### Example 5

FRET Assay

Europium chelate-labeled PPARs were prepared as follows. Fluorescently-labeled PPARs were prepared by incubation of the desired ligand binding domain with an equimolar concentration of LANCE™ M europium-(W8044) labeled streptavidin (Perkin Elmer Life Sciences) in 50 mM TRIS (pH 8), 50 mM NaCl, 1 mM CHAPS, 1 mM EDTA, 0.1 mg/ml BSA, and 10 mM DTT. After a 30 minute incubation at room temperature, excess biotin was added to block any residual unoccupied biotin binding sites and incubation was continued for a further 30 minutes.

Allophycocyanin-labeled RXRα LBD, CBP and N-CoR were prepared as follows. (APC)-labeled CBP, RXRα LBD, and NCoR ID-C peptides were prepared by incubation of equimolar concentrations of biotinylated-peptide and APC-labeled streptavidin (Molecular Probes, Eugene, Oreg.). After a 30 minute incubation at room temperature, excess biotin was added to block any residual unoccupied biotin binding sites and incubation was continued for a further 30 minutes.

Concentrations of europium-labeled PPAR receptor, APC-labeled, RXRα LBD, NCoR ID-C or CBP, and ligand were mixed in individual wells of 96-well plates. The concentration of the three PPAR subtypes was 10 nM. The concentration of CBP ranged from 25 nM to 150 nM the concentration of NCoR ranged from 100 nM to 250 nM; the concentration of RXRα LBD ranged from 4 nM to 12 nM The plates were incubated for at least 3 hours at room temperature. Samples were protected from light during the incubation period. Time-resolved fluorescence intensities were determined in a VICTOR™ 1420 Multilabel Counter. Plots of fluorescence intensity versus ligand concentration were constructed.

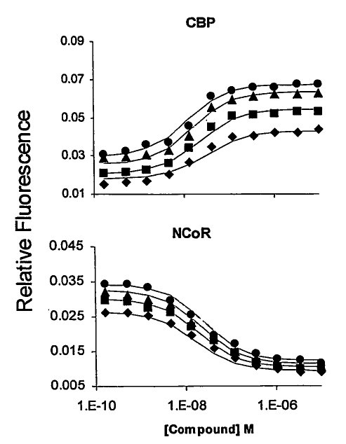

FIG. 4 illustrates the results of a GW1929 modulation of PPARγ cofactor complexes. The effect of GW1929 on 10 mM PPARγ LBD binding to CBP (57-454) or NCoR ID-C peptide were determined by time-resolved fluorimetry as described in methods. The relative fluorescence is the ratio of the fluorescence intensity at 665 nm and 610 nm. Concentrations of CBP were 25 nM (♦), 50 nM (▪), 100 nM (▴) and 150 nM (●). Concentrations of NCoR ID-C are 100 nM (♦), 150 nM (▪), 200 nM (▴) and 250 nM (●). The resulting curves were simultaneously fit to an interaction model described in Example 6.

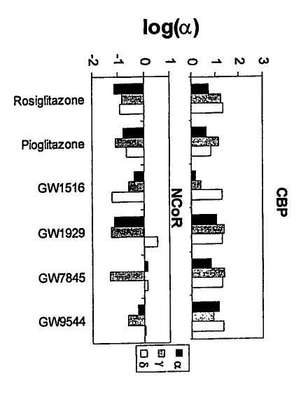

FIG. 5 illustrates the effect of ligands on coactivator and corepressor binding to PPAR subtypes determined by FRET. The fold increases in affinity (α) of various ligands on PPAR subtype binding to a fragment of CBP (57 to 454) or the NCoR ID-C peptide were determined by fluorescence energy transfer (FRET) as described in methods.

### Example 6

Interaction Model to Describe Ligand Modulation of Receptor-Cofactor Affinity

To describe ligand modulation of receptor-cofactor affinity, it is assume that receptor R, interacts with a cofactor X to form a complex XR, with an interaction constant K_{d}. The receptor can also interact with ligand, L to form a receptor ligand complex RL with an interaction constant of K_{L}. Formation of the ternary complex XRL, is also observed. The affinity of cofactor-receptor complex in the presence of (saturating) ligand changes by a factor of α when compared to the affinity in the absence of ligand. The affinity of receptor-ligand complex in the presence of (saturating) cofactor must also change by the same factor, α. The relevant binding equilibria are shown in equations 1-4.

$$\begin{matrix}{K}_{d} = \frac{R \cdot X}{XR} & ( 1 ) \\ {K}_{d} / α = \frac{RL \cdot X}{XRL} & ( 2 ) \\ {K}_{L} = \frac{R \cdot L}{RL} & ( 3 ) \\ {K}_{L} / α = \frac{XR \cdot L}{XRL} & ( 4 )\end{matrix}$$

R, X, and L are the free concentrations of receptor, cofactor, and ligand, respectively. The total receptor, cofactor, and ligand concentrations, R₀, X₀, and L₀ are described by equations 5-7. For a typical experiment, the cofactor and receptor are labeled with fluorophores so that receptor-cofactor interaction can be monitored by time-resolved energy transfer. In this case, the total cofactor-receptor complex (ligand bound and ligand free) is denoted as b (equation 8).
R₀=(R+RL+XR+XRL)=(R+RL+b) (5)
X₀=(X+XR+XRL)=(X+b) (6)
L₀=L+RL+XRL (7)
b=XR+XRL (8)
Equations 1, 2, and 8 may be combined as follows to provide an approximate mathematical solution for complex formation.

$$\begin{matrix}b = ( XR + XRL ) \\ = ( \frac{R \cdot X}{{K}_{d}} + \frac{α \cdot RL \cdot X}{{K}_{d}} ) \\ = \frac{X}{{K}_{d}} ( R + α \cdot RL )\end{matrix} \qquad ( 9 )$$

Substitution for X and R into equation 9 using the equalities in equations 6 and 5, respectively, gives:
K_{d}·b=(X₀−b)·(R₀−b+RL·(α−1)) (10)
If R0<<K_{L} and R0<<αK_{L}, then RL and XRL are small with respect to L₀, equation 3 may be approximated as:

$${K}_{L} \approx \frac{R \cdot {L}_{0}}{RL} = \frac{( {R}_{0} - RL - b ) \cdot {L}_{0}}{RL} \qquad ( 11 )$$

Solving equation 11 for RL and substituting this into equation 10 gives:

$$b \cdot {K}_{d} \cdot [ \frac{{K}_{L} + {L}_{0}}{{K}_{L} + α \cdot {L}_{0}} ] = ( {X}_{0} - b ) \cdot ( {R}_{0} - b ) \qquad ( 12 )$$

Equation 12 is a quadratic in b that can be solved for bound to give:

$$\begin{matrix}b = \frac{v - \sqrt{{υ}^{2} - 4 \cdot {R}_{0} {X}_{0}}}{2} where & ( 13 ) \\ υ = ( {X}_{0} + {R}_{0} + {K}_{d} \cdot [ \frac{{K}_{L} + {L}_{0}}{{K}_{L} + α \cdot {L}_{0}} ] ) & ( 14 )\end{matrix}$$

In order to convert complex formed, b, to an observed fluorescence signal, it is assumed that the background is constant. Alternatively, the background may be assumed to be proportional to the concentration of fluorescent cofactor in the well. In the case where background is constant. background signal is denoted as q₀. If q₁ is the factor to convert bound complex, in molar units, to fluorescence the fluorescence signal is given by equation 15.

$$F = {q}_{0} + {q}_{1} \cdot ( \frac{v - \sqrt{{υ}^{2} - 4 \cdot {R}_{0} {X}_{0}}}{2} ) \qquad ( 15 )$$

For convenience, all concentrations are expressed in units of nanomolar (nM). K_{d} and K_{L} are also expressed in units of nM while α is a unitless quantity. The background value, q₀ is expressed in arbitrary fluorescence units. In contrast, the quantity q₁ is expressed as fluorescence units per unit of concentration.

Experimental data were fit to equation 15 using a nonlinear least squares algorithm (typically using Microsoft Excel or SigmaPlot). Ligand and cofactor concentrations (L₀ and R₀, respectively) served as independent variables and observed fluorescence, F was the dependent variable. The total receptor concentration, R₀, was held constant and linear least squares estimates of K_{d}, K_{L}, α, q₀, and q₁ were obtained.

### Example 7

Mammalian Two-hybrid Assay

Expression plasmids for the VP16-human PPAR constructs were prepared by inserting amplified cDNAs encoding full length PPARα, PPARγ2 and PPARδ fused to amino acids 410-490 of the VP16 viral activation domain, into the expression vector pVP16 (Clonetech). GAL4-CBP (1-115), GAL4-NcoR (2012-2103), GAL4-NcoR (2239-2300) and GAL4-NcoR (1944-end) were generated by insertion of PCR amplified cDNAs encoding the indicated amino acids fused into a modified GAL4 DNA-binding domain (amino acids 1-147). The reporter plasmid was (UAS)₅-tk-SPAP, and the internal control plasmid for all transfections was β-galactosidase expression vector (pCH110, Amersham).

CV-1 cells were maintained in culture in DME high glucose medium supplemented with 10% FBS and 2 mM glutamine in a humidified incubator (5% CO₂ in air) at 37° C. Cells were harvested 72 hours prior to experimental use and placed in phenol red free D-MEM/F-12 medium with 15 mM HEPES supplemented with 10% charcoal/dextran-treated FBS (HyClone, Logan, Utah). The cells were harvested and seeded at 2.0×10⁴ cell per well in a 96-well plate the day prior to transfection. Cells were transfected for 16 hr using Lipofectamine (Life Technologies, Inc., Rockville, Md.) essentially according to the manufacturer's instructions. The total amount of DNA transfected into each well was 80 ng. Transfection mixtures contained 8 ng VP16-PPAR plasmid, 8 ng SPAP reporter, 25 ng of pCH110(β-galactosidase) control plasmid, 35 ng of pBluescript II KS+ (Clonetech) and 4 ng of either coactivator or corepressor plasmid. Transfection quantities for full length PPAR and cofactor plasmids were optimized to generate a signal that was half of the maximal activation. These conditions would allow for dissociation or association to be experimentally determined for each ligand evaluated. Stock solutions of drugs were prepated in DMSO and dilutions were made in phenol red-free D-MEM/F-12 with 15 mM HEPES supplemented with 10% charcoal stripped, delipidated, heat inactivated (62° C. for 35 min) bovine calf serum (Sigma, St. Louis, Mo.) such that the final concentration of DMSO was 0.1%. Transfection mixtures were aspirated and 100 μl, of medium containing drug was added to each well. Cells were incubated for 24 hours in the presence of drug. The medium was then sampled and assayed for alkaline phosphatase activity and the cell lysates were assayed for β-galactosidase activity to normalize for transfection efficiency. Plates were read on a Thermomax platereader (Molecular Devices) at 405 nM.

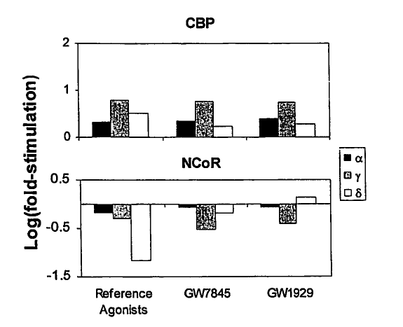

FIG. 6 shows the characterization of ligand effects on PPAR/cofactor interactions in a cell-based assay. Complex formation was evaluated using a mammalian two-hybrid interaction assay with fill-length PPAR receptors and cofactor fragments as described in methods. The effect of 10 μM of the indicated compound on VP16 full length PPARα, PPARγ or PPARδ binding to GAL4-CBP or GAL4-NCoR 2239 to 2300 is shown. Reference agonists were GW9820x for PPARα, pioglitazone for PPARγ and GW1516 for PPARδ. Values determined for each ligand were significantly different from vehicle at p<0.01.

### Example 8

Fluorescence Energy Transfer Assay for LXR

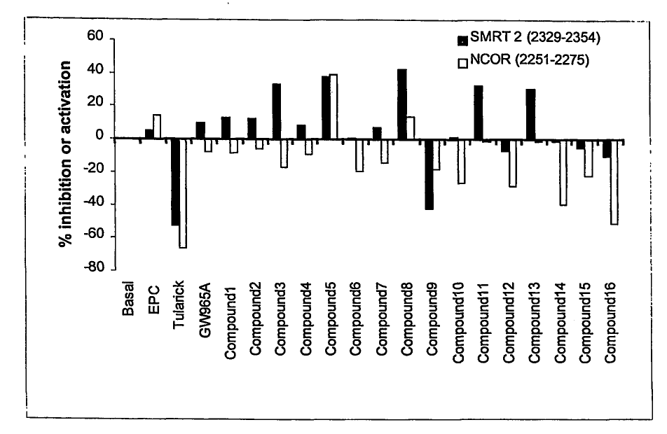

1 ul of 2 uM biotinylated co-repressor peptides in 100% DMSO were added to wells of a black 384 well plate. 25 ul of europium labeled streptavidin (40 nM) was then added to the plate and allowed to equilibrate for 30 min. A solution of 20 nM biotiylated LXR alpha LBD labeled with 20 nM streptavidin APC and subsequently blocked with a 40 fold molar excess of biotin was allowed to equilibrate with test compound at a final concentration of 2 uM for 30 minuets. An equal volume of APC-Labeled LXRα and test compound was added to the plate containing peptide and europium labeled streptavidin. The plates were incubated for at least 3 hours at room temperature. Time-resolved fluorescence intensities were determined in a Victor™ 1420 Multilabel Counter. Fluorescence intensity ratio (intensity at 665 nm/intensity at 620 mn) was determined and these values were normalized to % activation or inhibition. A bar graph, shown in FIG. 7, was constructed, each point was done in quadruplicate at each co-repressor concentration.

1. An isolated corepressor peptide or fragment thereof derived from a nuclear receptor interacting motif of corepressor Nuclear Receptor Corepressor (NCoR) or Silencing Mediator of Retinoid and Thyroid receptors (SMRT) which binds to nuclear receptor ligand binding domains.

2. The isolated corepressor peptide of claim 1 comprising GHSFADPASNLGLEDIIRKALMGSF (SEQ ID NO:4) or GTGLMTYRSQAVQEHASTNMGLEAIIRKALMGKYDQWEE (SEQ ID NO:5), or fragments thereof.

3. A method for evaluating selectivity of a ligand for a nuclear receptor comprising assessing a ligand's ability to increase or decrease binding of a coactivator peptide to the nuclear receptor and assessing the ligand's ability to displace from the nuclear receptor or bind to the nuclear receptor a corepressor peptide of claim 1.

4. The method of claim 3 wherein the nuclear receptor is a PPAR of subtype PPARα, PPARδ or PPARγ or LXRα, or LXRβ.

5. The method of claim 3 wherein the ligand's ability to increase or decrease binding of a co-activator peptide and to increase or decrease binding of a corepressor peptide is assessed by fluorescence polarization (FP), fluorescent resonance energy transfer (FRET), AlphaScreen, or surface plasmon resonance (SPR).

6. The method of claim 3 further comprising assessing the ligand's ability to increase or inhibit binding of a heterodimeric partner of the nuclear receptor.

7. The method of claim 6 wherein the nuclear receptor is a PPAR of subtype PPARα, PPARδ or PPARγ and the heterodimeric partner is RXR.

8. The method of claim 3 further comprising assessing the ligand's activity in a standard nuclear receptor-ligand binding assay, a cell-based reporter assay or a disease-specific cell-based assay.

9. A method for quantitatively profiling selectivity of a test compound for a nuclear receptor comprising assessing a test compound's ability to increase or decrease binding of a nuclear receptor to a coactivator peptide. and assessing the test compound's ability to displace from the nuclear receptor or bind to the nuclear receptor a co-repressor peptide of claim 1.

10. The method of claim 9 further comprising assessing the test compound's ability to increase or inhibit binding of a heterodimeric partner of the nuclear receptor.

11. The method of claim 10 wherein the nuclear receptor is a PPAR of subtype PPARα, PPARδ or PPARγ and the heterodimeric partner is RXR.
